(19) United States
(12) Patent Application Publication
(10) Pub. No.: US 2026/0256534 A1
(43) Pub. Date: Sep. 3, 2026

(54) ROBOTIC SURGICAL SYSTEM AND METHOD FOR USING ARTIFICIAL INTELLIGENCE TO GENERATE SIMULATION AND DECISION SUPPORT FOR TELEOPERATION

(71) Applicant: Auris Health, Inc., Santa Clara, CA (US)

(72) Inventors: Danyal FER, Santa Clara, CA (US); Ye TIAN, Santa Clara, CA (US); Pablo Garcia KILROY, Santa Clara, CA (US)

(21) Appl. No.: 19/655,442

(22) Filed: Apr. 22, 2026

Related U.S. Application Data
(63) Continuation of application No. PCT/IB2024/060619, filed on Oct. 28, 2024.
(60) Provisional application No. 63/595,052, filed on Nov. 1, 2023.

(51) Int. Cl. A61B 34/37; A61B 34/00; A61B 34/10; A61B 34/30; A61B 34/35; A61B 90/00

(52) U.S. Cl. CPC A61B 34/37; A61B 34/10; A61B 34/25; A61B 34/35; A61B 90/37; A61B 2034/105; A61B 2034/107; A61B 2034/256; A61B 2034/258; A61B 2034/306; A61B 2090/371

(57) ABSTRACT

A robotic surgical system and method for using artificial intelligence to generate simulation and decision support for teleoperation are provided. In one embodiment, the robotic surgical system provides a surgical simulation model with input about a surgical field of a patient and signals generated in response to user-manipulation of the plurality of user input devices to perform a surgical task on the patient. The surgical simulation model generates and displays a video that represents a simulated performance of the surgical task. Other embodiments are provided.

### CROSS-REFERENCE TO RELATED APPLICATIONS

The present patent application is a bypass continuation and claims priority to PCT Application No. PCT/IB 2024/060619, filed Oct. 28, 2024, (Docket No. 010336-20020B-WO), which claims the benefit of the filing date under 35 U.S.C. § 119(e) of Provisional U.S. patent application Ser. No. 63/595,052, filed Nov. 1, 2023, which is hereby incorporated by reference.

### TECHNICAL FIELD

The following embodiments generally relate to the field of robotic surgery and more specifically to the use of artificial intelligence with a robotic surgical system.

### BACKGROUND

Minimally-invasive surgery (MIS), such as laparoscopic surgery, involves techniques intended to reduce tissue damage during a surgical procedure. For example, laparoscopic procedures typically involve creating a number of small incisions in the patient (e.g., in the abdomen), and introducing one or more surgical instruments (e.g., an end effector, at least one camera, etc.) through the incisions into the patient. The surgical procedures may then be performed using the introduced surgical instruments, with the visualization aid provided by the camera.

Generally, MIS provides multiple benefits, such as reduced patient scarring, less patient pain, shorter patient recovery periods, and lower medical treatment costs associated with patient recovery. In some embodiments, MIS may be performed with robotic systems that include one or more robotic arms for manipulating surgical instruments based on commands from an operator. A robotic arm may, for example, support at its distal end various devices, such as surgical end effectors, imaging devices, cannulae for providing access to the patient's body cavity and organs, etc.

### BRIEF DESCRIPTION OF THE DRAWINGS

FIG. 1A depicts an example of an operating room arrangement with a robotic surgical system and a user console of an embodiment.

FIG. 1B is a schematic illustration of one exemplary variation of a robotic arm manipulator, tool driver, and cannula with a surgical tool of an embodiment.

FIG. 1C is a schematic illustration of an exemplary user console of an embodiment.

FIG. 2 is a schematic illustration of an exemplary variation of a user console for a robotic surgical system of an embodiment in communication with one or more third party devices.

FIG. 3 is a schematic of a surgical robotic platform of an embodiment with a graphical user interface (GUI) module, where the surgical robotic platform is in communication with multiple medical data resources.

FIGS. 4A and 4B are perspective and longitudinal cross-sectional views, respectively, of one exemplary variation of a handheld user input device of an embodiment.

FIG. 5 is a flowchart of a method of an embodiment for using artificial intelligence to generate simulation and decision support for teleoperation.

FIG. 6 is a block diagram of a generalized adversarial network and a variational autoencoder for use in an embodiment.

### DETAILED DESCRIPTION

Non-limiting examples of various aspects and variations of the embodiments are described herein and illustrated in the accompanying drawings.

### Robotic Surgical System Overview

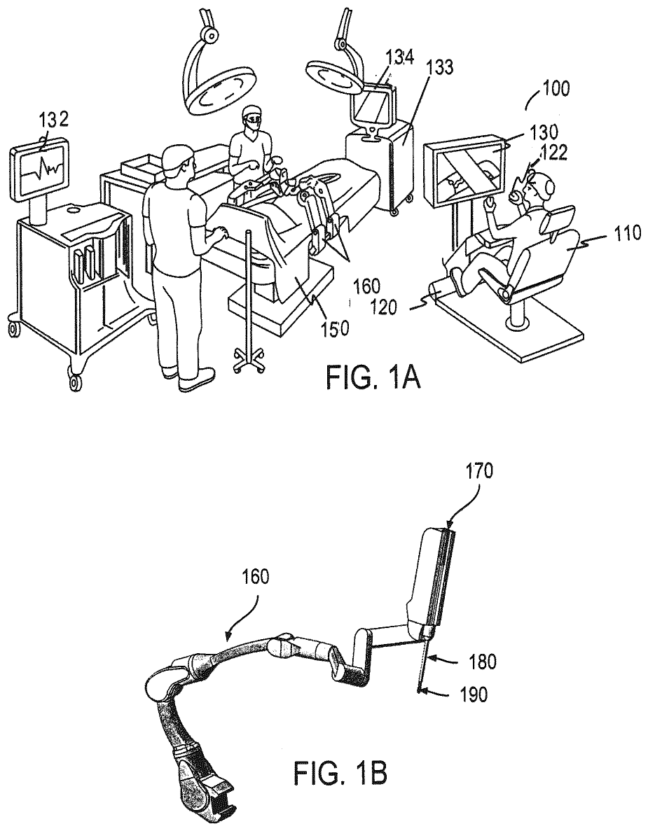

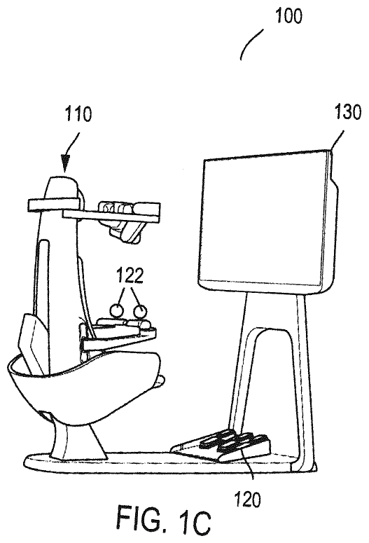

FIG. 1A is an illustration of an exemplary operating room environment with a robotic surgical system. Generally, as shown in FIG. 1A, the robotic surgical system includes a user console 100 (sometimes referred to herein as the “surgeon bridge” or “bridge”), a control tower 133, and one or more robotic arms 160 located at a robotic platform (e.g., table, bed, etc.), where surgical instruments (e.g., with end effectors) are attached to the distal ends of the robotic arms 160 for executing a surgical procedure. The robotic arms 160 are shown as a table-mounted system, but in other configurations, one or more robotic arms may be mounted to a cart, ceiling or sidewall, or other suitable support surface.

As further illustration, as shown in the exemplary schematic of FIG. 1B, a robotic surgical system may include at least one robotic arm 160 and a tool driver 170 generally attached to a distal end of the robotic arm 160. A cannula 180 coupled to the end of the tool driver 170 may receive and guide a surgical instrument 190 (e.g., end effector, camera, etc.). Furthermore, the robotic arm 160 may include a plurality of links that are actuated so as to position and orient the tool driver 170, which actuates the surgical instrument 190.

Generally, as shown in FIG. 1A, the user console 100 may be used to interface with the robotic surgical system 150. A user (such as a surgeon or other operator) may use the user console 100 to remotely manipulate the robotic arms 160 and/or surgical instruments (e.g., in tele-operation). The user console 100 may be located in the same operating room as the robotic system 150, as shown in FIG. 1A. In other embodiments, the user console 100 may be located in an adjacent or nearby room, or tele-operated from a remote location in a different building, city, or country. In one example, the user console 100 may comprise a seat 110, foot-operated controls (pedals) 120, one or more handheld user input devices 122, and at least one user display 130 configured to display, for example, a view of the surgical site inside a patient (e.g., captured with an endoscopic camera), and/or other surgical or medical information.

In the exemplary user console shown in FIG. 1C, a user located in the seat 110 and viewing the user display 130 may manipulate the foot-operated controls 120 and/or handheld user input devices 122 to remotely control the robotic arms 160 and/or surgical instruments mounted to the distal ends of the arm. The foot-operated controls 120 and/or handheld user input devices 122 may additionally or alternatively be used to control other aspects of the user console 100 or robotic system 150. For example, in variations in which the user generally controls (at any given time) a designated “left-hand” robotic arm/instrument and a designated “right-hand” robotic arm/instrument, the foot-operated controls 120 may enable a user to designate from among a larger group of available robotic arms/instruments which robotic arms/instruments comprise the “left-hand” and “right-hand” robotic arm/instruments (e.g., via toggle or rotation in selection among the available robotic arms/instruments). Other examples include adjusting or configuring the seat 110, the foot-operated controls 120, the user input devices 122, and/or the user display 130.

In some variations, a user may operate the surgical robotic system in an “over the bed” (OTB) mode, in which the user is at the patient's side and simultaneously manipulating a robotically-driven instrument/end effector attached thereto (e.g., with a handheld user input device 122 held in one hand) and a manual laparoscopic tool. For example, the user's left hand may be manipulating a handheld user input device 122 to control a robotic surgical component, while the user's right hand may be manipulating a manual laparoscopic tool. Accordingly, in these variations, the user may perform both robotic-assisted MIS and manual laparoscopic surgery on a patient.

During an exemplary procedure or surgery, the patient is prepped and draped in a sterile fashion, and anesthesia may be achieved. Initial access to the surgical site may be performed manually with the robotic system 150 in a stowed configuration or withdrawn configuration to facilitate access to the surgical site. Once access is completed, initial positioning and/or preparation of the robotic system may be performed. During the surgical procedure, a surgeon or other user in the user console 100 may utilize the foot-operated controls 120, user input devices 122, and/or other suitable controls to manipulate various end effectors and/or imaging systems to perform the procedure. Manual assistance may be provided at the procedure table by other personnel, who may perform tasks including but not limited to retracting tissues, or performing manual repositioning or tool exchange involving one or more robotic arms 160. Other personnel may be present to assist the user at the user console 100. Medical and surgery-related information to aid other medical personnel (e.g., nurses) may be provided on additional displays such as a display 134 on a control tower 133 (e.g., control system for the robotic surgical system) and/or a display 132 located bedside proximate the patient. For example, as described in further detail herein, some or all information displayed to the user in the user console 100 may also be displayed on at least one additional display for other personnel and/or provide additional pathways for inter-personnel communication. When the procedure or surgery is completed, the robotic system 150 and/or user console 100 may be configured or set in a state to facilitate one or more post-operative procedures, including but not limited to robotic system cleaning and/or sterilization, and/or healthcare record entry or printout, whether electronic or hard copy, such as via the user console 100.

In some variations, the communication between the robotic system 150, the user console 100, and any other displays may be through the control tower 133, which may translate user commands from the user console 100 to robotic control commands and transmit them to the robotic system 150. The control tower 133 may transmit status and feedback from the robotic system 150 back to the user console 100 (and/or other displays). The connections between the robotic system 150, the user console 100, other displays, and the control tower 133 may be via wired and/or wireless connections, and may be proprietary or performed using any of a variety of data communication protocols. Any wired connections may be built into the floor and/or walls or ceiling of the operating room. The robotic surgical system may provide video output to one or more displays, including displays within the operating room as well as remote displays accessible via the Internet or other networks. The video output or feed may be encrypted to ensure privacy, and all or one or more portions of the video output may be saved to a server, an electronic healthcare record system, or other suitable storage medium.

In some variations, additional user consoles 100 may be provided, for example to control additional surgical instruments, and/or to take control of one or more surgical instruments at a primary user console. This will permit, for example, a surgeon to take over or illustrate a technique during a surgical procedure with medical students and physicians-in-training, or to assist during complex surgeries requiring multiple surgeons acting simultaneously or in a coordinated manner.

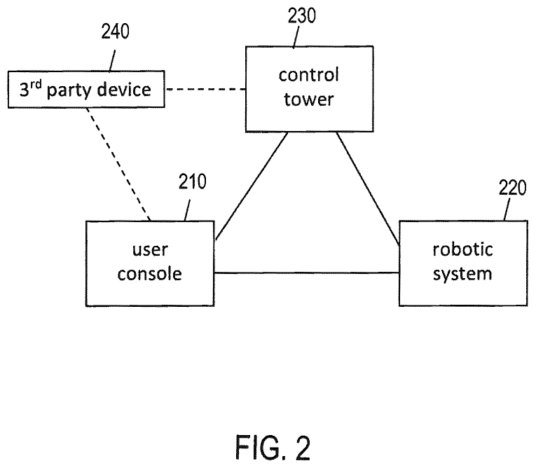

In some variations, as shown in the schematic illustration of FIG. 2, one or more third party devices 240 may be configured to communicate with the user console 210 and/or other suitable portions of the robotic surgical system. For example, as described elsewhere herein, a surgeon or other user may sit in the user console 210, which may communicate with the control tower 230 and/or robotic instruments in a robotic system 220. Medical data (e.g., endoscopic images, patient vitals, tool status, etc.) may be displayed at the user console 210, the control tower 230, and/or other displays. At least a subset of the surgical and other medical-related information may furthermore be displayed at a third party device 240, such as a remote computer display that is viewed by a surgical collaborator in the same room or outside the room. Other communication, such as teleconferencing with audio and/or visual communication, may further be provided to and from the third party device. The surgical collaborator may be, for example, a supervisor or trainer, a medical colleague (e.g., radiologist), or other third party who may, for example, view and communicate via the third party device 240 to assist with the surgical procedure.

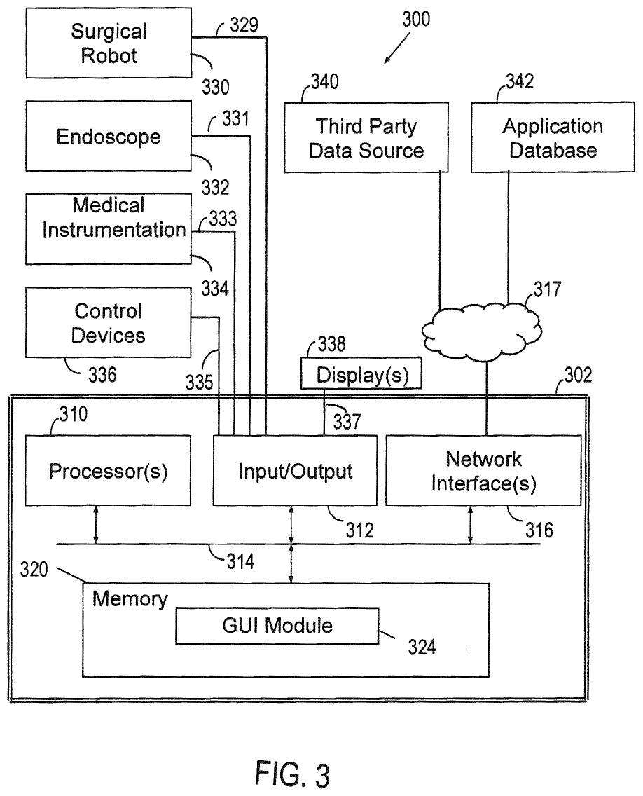

FIG. 3 is a schematic illustration of an exemplary variation of a system 300 including a robotic surgical system and its interaction with other devices and parties. Although a particular architecture of the various connected and communicating systems is depicted in FIG. 3, it should be understood that in other variations, other suitable architectures may be used and the arrangement shown in FIG. 3 is for illustrative purposes. The system 300 may include a surgical robotic platform 302 that facilitates the integration of medical data from discrete medical data resources generated from a variety of parties. Data from the discrete medical data resources may, for example, be used to form temporally coordinated medical data. Multi-panel displays of the temporally coordinated medical data may be configured and presented, as described further herein.

The platform 302 may be, for example, a machine with one or more processors 310 connected to one or more input/output devices 312 via a bus 314. The at least one processor may, for example, include a central processing unit, a graphics processing unit, an application specific integrated circuit, a field programmable logic device or combinations thereof.

The surgical robotic platform 302 may include one or more input ports to receive medical data from discrete medical data resources. For example, a surgical robot port 329 may receive surgical robot data from a surgical robot 330. Such data may, for example, include position data or other suitable status information. An imaging port 331 may receive imaging data from an imaging device 332, such as an endoscope, that is configured to capture images (e.g., still images, video images) of a surgical site. The endoscope may, for example, be inserted through a natural orifice or through an aperture in a surgical patient. As another example, one or more medical instrumentation ports 333 may receive patient vital information from medical instrumentation 334 (e.g., a pulse oximeter, electrocardiogram device, ultrasound device and/or the like). Additionally, as another example, one or more user control data ports 335 may receive user interaction data from one or more control devices that receive user inputs from a user for controlling the system. For example, one or more handheld user input devices, one or more foot pedals, and/or other suitable devices (e.g., eye tracking, head tracking sensors) may receive user inputs.

The surgical robotic platform 302 may further include one or more output ports 337 configured for connection to one or more displays 338. For example, the displays 338 may include an open display (e.g., monitor screen) in a user console, an immersive display or head-mounted device with a display, on supplemental displays such as on a control tower display (e.g., team display), a bedside display (e.g., nurse display), an overhead “stadium” style screen, etc. For example, the graphical user interface disclosed herein may be presented on one or more displays 338. The one or more displays 338 may present three-dimensional images. In some variations, the one or more displays 338 may include a touchscreen. The one or more displays 138 may be a single display with multiple panels, with each panel presenting different content. Alternatively, the one or more displays 138 may include a collection of individual displays, where each individual display presents at least one panel.

In some variations, a network interface 316 may also be connected to the bus 314. The network interface 316 may, for example, provide connectivity to a network 317, which may be any combination of one or more wired and/or wireless networks. The network 317 may, for example, help enable communication between the surgical robotic platform 302 and other data sources or other devices. For example, one or more third party data sources 340 may also be connected to the network 317. The third party source 340 may include a third party device (e.g., another computer operated by a third party such as another doctor or medical specialist), a repository of video surgical procedure data (e.g., which may be relevant to a procedure being performed by a surgeon), or other suitable source of additional information related to a surgical procedure. For example, the third party device data may be ported to a panel that is displayed to a surgeon before, during or after a procedure.

As another example, one or more application databases 342 may be connected to the network 317 (or alternatively, stored locally within a memory 320 within the surgical robotic platform 302). The application database 342 may include software applications (e.g., as described in further detail below) that may be of interest to a surgeon during a procedure. For example, a software application may provide access to stored medical records of a patient, provide a checklist of surgical tasks for a surgical procedure, perform machine vision techniques for assisting with a procedure, perform machine learning tasks to improve surgical tasks, etc. Any suitable number of applications may be invoked. Information associated with an application may be displayed in a multi-panel display or other suitable display during a procedure. Additionally or alternatively, information provided by one or more applications may be provided by separate resources (e.g., a machine learning resource) otherwise suitably in communication with the surgical robotic platform 302.

In some variations, one or more of the software applications may run as a separate process that uses an application program interface (API) to draw objects and/or images on the display. APIs of different complexities may be used. For example, a simple API may include a few templates with fixed widget sizes and locations, which can be used by the GUI module to customize text and/or images. As another example, a more complex API may allow a software application to create, place, and delete different widgets, such as labels, lists, buttons, and images.

Additionally or alternatively, one or more software applications may render themselves for display. This may, for example, allow for a high level of customization and complex behavior for an application. For example, this approach may be implemented by allowing an application to pass frames that are rendered by a graphical user interface (GUI) module 324, which can be computer-readable program code that is executed by the processor 310. Alternatively, an image buffer may be used as a repository to which an application renders itself.

In some variations, one or more software applications may run and render themselves independent of the GUI module 324. The GUI module may still, however, launch such applications, instruct the application or the operating system where the application is to be positioned on the display, etc.

As another approach, in some variations, one or more applications may run completely separate from the GUI rendered by the GUI module. For example, such applications may have a physical video connection and data connection to the system (e.g., through suitable input/output devices, network, etc.). The data connection may be used to configure video feed for an application to be the appropriate pixel dimensions (e.g., full screen, half screen, etc.).

As shown in FIG. 3, in some variations, a memory 320 may also be connected to the bus 314. The memory 320 may be configured to store data processed in accordance with embodiments of the methods and systems described herein.

In some variations, the memory 320 may be configured to store other kinds of data and/or software modules for execution. For example, a user console may include a memory 320 that stores a GUI module 324 with executable instructions to implement operations disclosed herein. The GUI module may, for example, combine and aggregate information from various software applications and/or other medical data resources for display. In some exemplary variations, one or more software applications may be incorporated into base code of the GUI module, such that the module draws graphics and displays text in the appropriate location on the display. For example, the module may fetch the images from a database, or the images may be pushed to the interface from an instrument (e.g., endoscopic camera) in the operating room, via a wired or wireless interface.

In some variations, medical data may be collected from discrete medical data resources (e.g., surgical robot 330, endoscope 332, medical instrumentation 334, control devices 336, third party data source 340, application database 342, etc.). Additionally, at least some of the medical data may be temporally coordinated such that, when necessary, time sensitive information from different medical data resources is aligned on a common time axis. For example, surgical robot position data may be time coordinated with endoscope data, which is coordinated with operator interaction data from control devices. Similarly, a networked resource, such as information provided by one or more software applications, may be presented at an appropriate point in time along with the other temporally coordinated data. Multi-panel displays, and/or other suitable displays, may be configured to communicate medical information (e.g., including the temporally coordinated medical data) as part of a graphical user interface (GUI).

Various exemplary aspects of a GUI for a robotic surgical system are described herein. In some variations, the GUI may be displayed in a multi-panel display at a user console that controls the robotic surgical system. Additionally or alternatively, the GUI may be displayed at one or more additional displays, such as at a control tower for the robotic surgical system, at a patient bedside, etc. Generally, the GUI may provide for more effective communication of information to a user in the user console and/or other personnel, as well as for more effective communication and collaboration among different parties involved in a surgical procedure, as further described below.

### Graphical User Interface (GUI) Interaction

In one embodiment, the GUI is displayed on a display 130 in a user console 100 that is used to control the robotic surgical system 150 (e.g., by a surgeon), and at least some of interactive graphical objects displayed on the display 130 may be controlled, selected, or otherwise interacted with via one or more user controls that are also used to control an aspect of the surgical system (e.g., surgical instrument). For example, a user may use one or more handheld user input devices 122 and/or one or more foot pedals 120 to selectively control an aspect of the robotic surgical system 150 and selectively interact with the GUI. By enabling control of both the robotic surgical system 150 and the GUI with the same user controls, the user may advantageously avoid having to switch between two different kinds of user controls. Enabling the user to use the same input devices to control the robotic system 150 and the GUI streamlines the surgical procedure and increases efficiency, as well as helps the user maintain sterility throughout a surgical procedure.

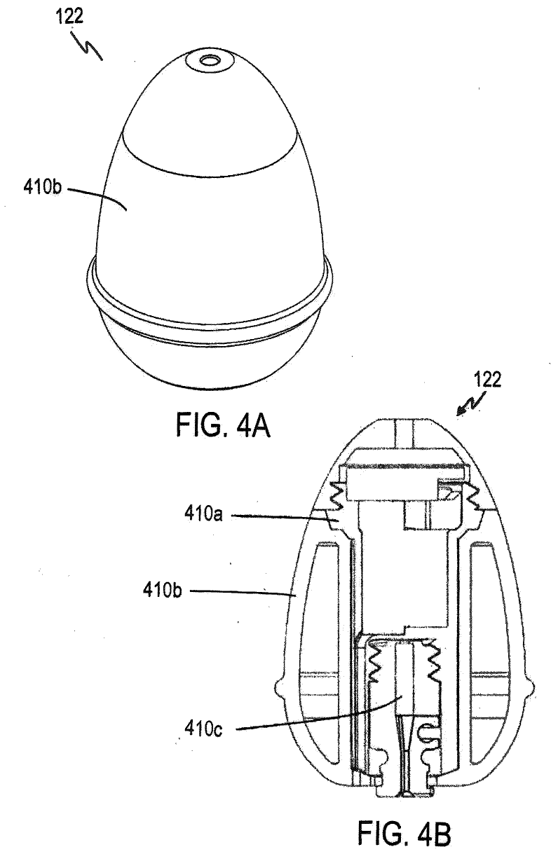

As shown generally in FIGS. 4A and 4B, an exemplary variation of a handheld user input device 122 for controlling a robotic system may include a member 410, a housing 420 at least partially disposed around the member 410 and configured to be held in the hand of a user, and a tracking sensor system 440 configured to detect at least position and/or orientation of at least a portion of the device. The housing 420 may be flexible (e.g., made of silicone). In some instances, the detected position and/or orientation of the device may be correlatable to a control of the robotic system. For example, the user input device 122 may control at least a portion of a robotic arm, an end effector or tool (e.g., graspers or jaws) coupled to a distal end of the robotic arm, a GUI, or other suitable aspect or feature of the robotic surgical system 150. Additionally, in some instances, the detected position and/or orientation of the device 122 may be correlatable to a control of a GUI. Furthermore, in some variations, the user input device 122 may include one or more sensors for detecting other manipulations of the user input device 122, such as squeezing of the housing 420 (e.g., via one or more pressure sensors, one or more capacitive sensors, etc.).

Generally, a user interface for controlling a robotic surgical system may include at least one handheld user input device 122, or may include at least two handheld user input devices 122 (e.g., a first user input device to be held by a left hand of the user, and a second user input device to be held by a right hand of the user), or any suitable number. Each user input device 122 may be configured to control one or more different aspects or features of the robotic system. For example, a user input device held in the left hand of the user may be configured to control an end effector represented on a left side of a camera view provided to the user, while a user input device held in the right hand of the user may be configured to control an end effector represented on a right side of the camera view.

In some variations, the handheld user input device 122 may be a groundless user input device configured to be held in the hand and manipulated in free space. For example, the user input device 122 may be configured to be held between the fingers of a user, and moved about freely (e.g., translated, rotated, tilted, etc.) by the user as the user moves his or her arms, hands, and/or fingers. Additionally or alternatively, the handheld user input device 122 may be a body-grounded user input device, in that the user input device 122 may be coupled to a portion of the user (e.g., to fingers, hand, and/or arms of a user) directly or via any suitable mechanism such as a glove, hand strap, sleeve, etc. Such a body-grounded user input device may still enable the user to manipulate the user input device in free space. Accordingly, in variations in which the user input device 122 is groundless or body-grounded (as opposed to permanently mounted or grounded to a fixed console or the like), the user input device 122 may be ergonomic and provide dexterous control, such as by enabling the user to control the user input device with natural body movements unencumbered by the fixed nature of a grounded system.

The handheld user input device 122 may include wired connections that, for example, may provide power to the user input device 122, carry sensor signals (e.g., from the tracking sensor assembly and/or other sensors such as a capacitive sensor, optical sensor, etc. Alternatively, the user input device may be wireless as shown in FIG. 4A and communicate commands and other signals via wireless communication such as radiofrequency signals (e.g., WiFi or short-range such as 400-500 mm range, etc.) or other suitable wireless communication protocol such as Bluetooth. Other wireless connections may be facilitated with optical reader sensors and/or cameras configured to detect optical markers on the user input device 122 infrared sensors, ultrasound sensors, or other suitable sensors.

The handheld user input device may include a clutch mechanism for switching between controlling a robotic arm or end effector and controlling a graphical user interface, etc., and/or between other control modes. One or more of the various user inputs described in further detail below may, in any suitable combination, function as a clutch. For example, touching a gesture touch region of the device, squeezing the housing, flicking or rotating the user input device, etc. may function to engage a clutch. As another example, a combination of squeezing and holding the user input device, and rotating the user input device, may function as a clutch. However, any suitable combination of gestures may function as a clutch. Additionally or alternatively, user input to other user input devices (e.g., foot pedal assembly) may, alone or in combination with user input to a handheld user input device, function as a clutch.

In some variations, engagement and disengagement of a clutch mechanism may enable transition between use of a handheld user input device as a control for the robotic system and use of the handheld user input device as a control for the GUI (e.g., to operate a cursor displayed on the screen). When a clutch mechanism is engaged such that the user input devices are used to control the GUI, positions or poses of the robotic arms may be substantially locked in place to “pause” operation of the robotic system, such that subsequent movement of the user input devices while the clutch is engaged will not inadvertently cause movement of the robotic arms.

### Use of Artificial Intelligence to Generate Simulation and Decision Support for Teleoperation

In another embodiment, the robotic surgical system is configured with artificial intelligence to run a simulation of that generates and displays a video that represents a simulated performance of a surgical task at a surgical field of a patient before the surgical task is actually performed on the patient. Such a simulation can be helpful in situations where the surgeon/surgeon bridge is located remotely from the robotic arms, such as when the surgeon/surgeon bridge is on Earth, and the robotic arms/patient are not on Earth (e.g., in a spaceship or on another planet).

The passage of the NASA Transition Authorization Act has affirmed the goal of a crewed mission to Mars by 2033. Multiple private and governmental space organizations anticipate human space travel beyond low-earth orbit. Crew health-related research estimates up to an 18% cumulative likelihood of a surgical emergency for a three-year mission. Previous work has focused on crew selection and training as a preventative measure. However, the resources and individual training required to have a complete and independent surgical capability for long-duration space travel are not presently available without significant innovations. Regardless of the robotics and tools under evaluation, on-board surgical expertise will be lacking and, thus, expertise must be brought to the crew. Presently for low-earth orbit, expertise is brought to astronauts through remote guidance from the ground. In deep space, expertise is separated from the crew by latency, with signal delays in excess of 50 minutes.

Because of these significant latencies, a surgeon on Earth will not timely be able to see the consequences of the surgical actions he is initiating at the surgical bridge on Earth. By using artificial intelligence to display a simulated performance of the surgical task, the surgeon can see a prediction of the consequences of his actions. If he is satisfied with the prediction, he can approve the transmission of the inputs provided to the surgical model to the actual surgical robot, so it can perform the actions on the patient. Medical personnel (e.g., a less-experience surgeon) located with the patient can be ready to handle any unexpected complications.

While this problem exists in space, the solution also has applicability terrestrially where both the surgeon and the patient are on Earth (e.g., in military, rural. and low-specialty-expertise environments). For example, a remote/teleoperation situation can occur when the surgeon is in one city, and the patient (and perhaps a less-experienced surgeon) is in another city, or even when the surgeon and patient are located in different rooms in the same city or building. Further, the surgical simulation model can also have applicability when the surgeon and patient are local to one another (e.g., in the same operating room). In the operating room, there a large number of sensors and data collection devices that can help the surgeon interpret the surgical field. This requires the surgeon to rely on his own experience in making critical surgical decisions, which can lead to complications causing patient harm. With these embodiments, the surgical simulation model can alert the surgeon to a possible complication to his intended actions, allowing the surgeon to change actions before the complication occurs.

The following embodiments present a predictive model that is able to predict future motions of instruments and their end effect on tissues effectively and accurately to predict future states of the surgical field. In one embodiment, artificial intelligence is used through a predictive model that may aid in the guidance of surgical procedures. This model can predict both the motion of surgical tools, as well as their effects on the patient. This embodiment can allow the surgeon to operate in a simulation to evaluate a specific surgical task prior to performing the task in reality.

The following paragraphs and accompanying drawings will discuss one possible implementation. It should be understood that other implementations are possible.

In general and as discussed above in conjunction with FIG. 2, in one embodiment, the robotic surgical system comprises a user console (surgeon bridge) 210 that comprises a display device and a plurality of user input devices (e.g., pedals, handheld controllers, a touch screen, etc.). As the user manipulates the plurality of user input devices, the user console 210 provides signals that are generated by the manipulation to a control tower 230, which in turn communicates with the plurality of robotic arms 220. A robotic arm controller in the control tower 230 and/or tableside where the robotic arms 220 are located responds to the signals by moving the plurality of robotic arms 220.

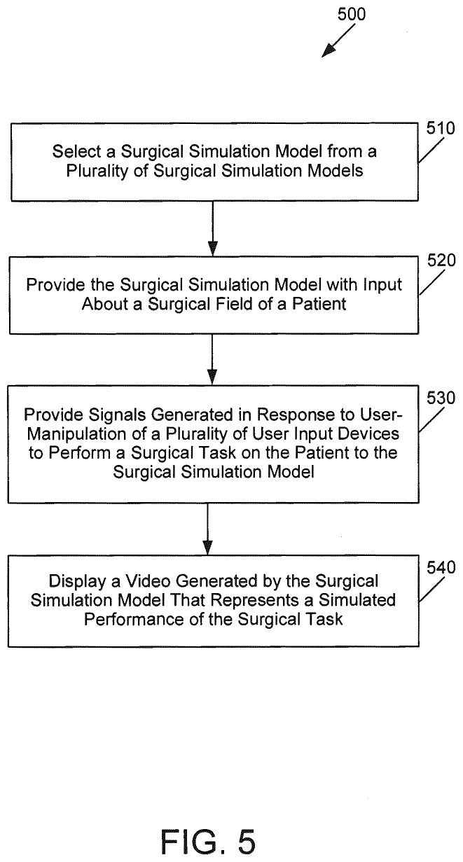

In addition, the system in this embodiment comprises a processor that is configured to perform various functions related to the surgical simulation model. In one embodiment, the processor is integrated in the user console 210 but can instead be located external to the user console 210, such as in a third-party device 240 or other location. In one embodiment, the processor is configured to execute computer-readable program code to carry out instructions to instantiate the surgical simulation model. In other embodiments, the processor is hardcoded to instantiate the surgical simulation model. The instruction code, whether embodied in software or hardware, can cause the processor to perform the algorithm shown in the flowchart 500 in FIG. 5.

As shown in FIG. 5, the processor selects a surgical simulation model from a plurality of surgical simulation models based information about the surgical procedure (e.g., gallbladder operation, appendix operation, etc.) and/or the patient (act 510).

Next, the processor provides a surgical simulation model with input about a surgical field of a patient (act 520). For example, the input can be a plurality of video frames of the surgical field captured by a first camera on one of the plurality of robotic arms, kinematic data regarding movement of the plurality of robotic arms, and a topographical three-dimensional representation of anatomy in the surgical field captured by a second camera (e.g., using structured light) on another one of the plurality of robotic arms. With these inputs, the surgical simulation model can have a baseline for its future predictions.

The processor can provide the surgical simulation model with additional data, such as, but not limited to, data from an electronic health record of the patient, data from an intraoperative sensor, imaging data of the patient, and previous video data of the patient. For example, if the data includes a CT scan of the patient, the surgical simulation model can use the location of blood vessels from the scan to more accurately position the blood vessels in the simulation. As another example, if the data indicates that the patient is on an anticoagulant, the simulation can more accurately reflect the volume of blood to expect from an incision or other changes in tissue.

Next, the processor provides signals generated in response to user-manipulation of the plurality of user input devices to perform a surgical task on the patient to the surgical simulation model instead of to the robotic arm controller (act 530). Because the signals are not provided to the robotic arm controller, movement of the user input devices does not result in actual movement of the robotic arms. Instead, the surgical simulation model takes these inputs and predicts, based on the earlier applied inputs and other information built into the model, the results of those action. More specifically, the prediction can be in the form of generated video frames which are displayed on the display device of the user console 210 and represent a simulated performance of the surgical task at the surgical field (act 540). Additionally, the processor can be configured to provide an indication of a likelihood of success of the surgical task, which can be in the form of a percentage or a general alert that a complication is possible or likely.

After viewing the simulated video and/or receiving the indication of likelihood of success, the surgeon can approve the simulated performance of the surgical task. In response to this approval, the processor can send the signals generated in response to user-manipulation of the plurality of user input devices (which were previously sent to the model) to the robotic arm controller to cause the plurality of robotic arms to perform the surgical task on the patient. If the surgeon is unsatisfied with the simulated performance, he can repeat the simulation until satisfied or make adjustment to some of the previously-recorded input signals.

Any suitable technology can be used to build the surgical simulation model. In one embodiment, the processor can take a plurality video frames captured by a camera viewing the surgical field (e.g., by an endoscopic camera on one of the robotic arms) as input to predict the next several video frames. To do that, a dataset can first be built by taking short video clips from some surgical phases (e.g., those that contain suturing activity). For example, four frames can be inputted into the model to output 15 frames as predictions. Then, a model with a recurrent generator can be trained on that dataset to synthesize new frames based on previous ones and some latent variables. For example, a combination of a generative adversarial network (GAN) and a variational autoencoder (VAE) can be used for this purpose.

The GAN-based training helps the model to make prediction that look real to human. At the same time, VAE-based training complements the model to keep diverse prediction. As mentioned above, besides the video captured from the surgical field, other information about the patient (e.g., data from the patient's electronic health record, data from an intraoperative sensor, imaging data, and previous video data) can be provided as a feed to a data set that leverages the collective experience of many to inform individual surgical decisions. To take such information into account in addition to the video feed from the patient to aid in the prediction, one or more autoencoders can be used to encode this information into some feature and concatenate it with latent variables Zt-1 during training and testing. In this way, the patient's information can also serve as prior information for the generator.

Using a generalized adversarial network and a variational autoencoder can allow the model to be able to predict futures frames with realism as well as diversity. The predictions can build in a hierarchical manner to predict more-complicated and longer surgical tasks. In order of increasing complexity, the model can predict primitives, maneuvers, tasks, surgical phases, and procedures. This model can also anticipate potential complications and the most clinically-adventitious path when driven by all surgical data including outcomes.

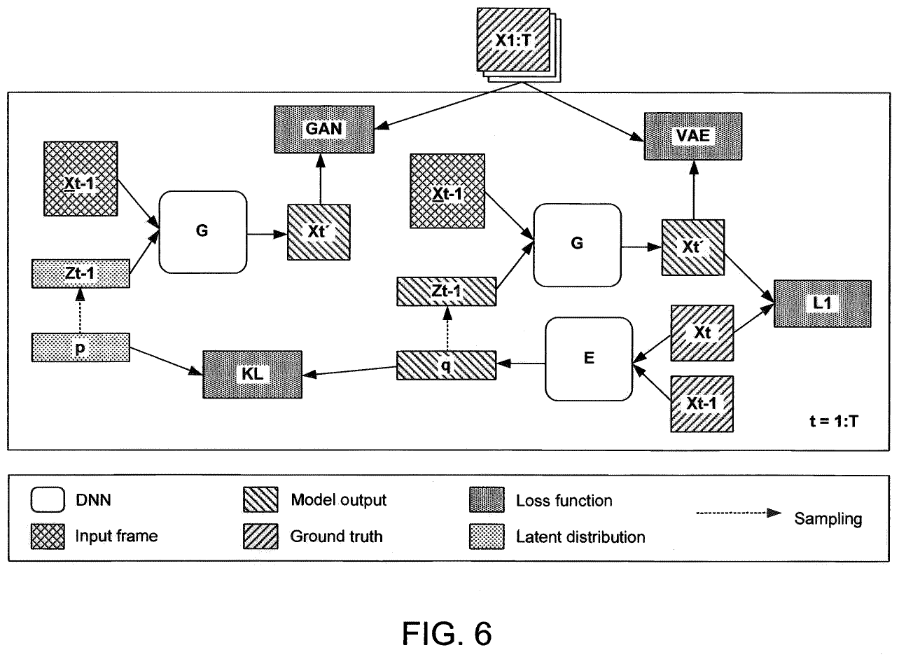

Returning to the drawings, FIG. 6 is a block diagram of a generalized adversarial network and a variational autoencoder that can be used with an embodiment to train the model. As shown in FIG. 6, deep neural networks (G, E) are represented in some blocks, previous frames are represented as Xt-1 in other blocks, p is a default distribution, and q is a distribution learned from an encoder network E. Zt-1 are latent variables sampled from p or q. The model output, ground truth frames, and the loss functions are represented separately. During training, a previous frame Xt-1 along with latent variables Zt-1 are input into the recurrent generator G. This recurrent generator is optimized to generate fake frames Xt′ that look real to some learned discriminator (e.g., adversarial loss with and without latent variables from the encoder) and are similar to the ground truth frames (l1 loss). The encoder E, at the same time and with two consecutive ground truth frames as input, is trained to make q similar to the default distribution (KL loss). Additionally, as mentioned above, patient information can be taken into account, and the autoencoder(s) can be used to encode this information into some feature and concatenate it with latent variables Zt-1 during training and testing. In this way, the patient's information can also serve as prior information for the generator.

Other Illustrative Embodiments include the following. Illustrative Embodiments for one type of claim (e.g., system, method, computer program, or computer readable storage medium) may be provided in other types (e.g., system as a method). Illustrative Embodiments for one set (e.g., Illustrative Embodiments 1-9) may be used in other sets.

Illustrative Embodiment 1. A robotic surgical system comprising: a user console comprising a display device and a plurality of user input devices; a plurality of robotic arms; a robotic arm controller configured to move the plurality of robotic arms in response to signals received from the user console that are generated in response to user-manipulation of the plurality of user input devices; and a processor configured to: provide a surgical simulation model with input about a surgical field of a patient; provide signals generated in response to user-manipulation of the plurality of user input devices to perform a surgical task on the patient to the surgical simulation model instead of to the robotic arm controller; and display, on the display device of the user console, a video generated by the surgical simulation model that represents a simulated performance of the surgical task.

Illustrative Embodiment 2. The robotic surgical system of Illustrative Embodiment 1, wherein the surgical simulation model uses a generalized adversarial network and a variational autoencoder.

Illustrative Embodiment 3. The robotic surgical system of any of Illustrative Embodiments 1-2, wherein the input about the surgical field comprises one or more of the following items: a plurality of video frames of the surgical field captured by a first camera on one of the plurality of robotic arms, kinematic data regarding movement of the plurality of robotic arms, and a topographical three-dimensional representation of anatomy in the surgical field captured by a second camera on another one of the plurality of robotic arms.

Illustrative Embodiment 4. The robotic surgical system of any of Illustrative Embodiments 1-3, wherein the processor is further configured to provide the surgical simulation model with one or more of the following items: data from an electronic health record of the patient, data from an intraoperative sensor, imaging data of the patient, and previous video data of the patient.

Illustrative Embodiment 5. The robotic surgical system of any of Illustrative Embodiments 1-4, wherein the processor is further configured to select the surgical simulation model from a plurality of surgical simulation models based information about a surgical procedure and/or the patient.

Illustrative Embodiment 6. The robotic surgical system of any of Illustrative Embodiments 1-5, wherein the processor is further configured to provide an indication of a likelihood of success of the surgical task.

Illustrative Embodiment 7. The robotic surgical system of any of Illustrative Embodiments 1-6, wherein the processor is further configured to: in response to receiving user approval of the simulated performance of the surgical task, provide the signals generated in response to user-manipulation of the plurality of user input devices to the robotic arm controller to cause the plurality of robotic arms to perform the surgical task on the patient.

Illustrative Embodiment 8. The robotic surgical system of any of Illustrative Embodiments 1-7, wherein the user console is on Earth, and the plurality of robotic arms are not on Earth.

Illustrative Embodiment 9. The robotic surgical system of any of Illustrative Embodiments 1-8, wherein the user console is in one building on Earth, and the plurality of robotic arms are in another building on Earth.

Illustrative Embodiment 10. The robotic surgical system of any of Illustrative Embodiments 1-9, wherein the processor is integrated in the user console.

Illustrative Embodiment 11. A method comprising: performing the following in a robotic surgical system comprising a plurality of robotic arms, the method comprising: providing a surgical simulation model with input about a surgical field of a patient; providing signals generated in response to user-manipulation of a plurality of user input devices to perform a surgical task on the patient to the surgical simulation model; and displaying a video generated by the surgical simulation model that represents a simulated performance of the surgical task.

Illustrative Embodiment 12. The method of Illustrative Embodiment 11, wherein the surgical simulation model uses a generalized adversarial network and a variational autoencoder.

Illustrative Embodiment 13. The method of any of Illustrative Embodiments 11-12, wherein the input about the surgical field comprises one or more of the following items: a plurality of video frames of the surgical field captured by a first camera on one of the plurality of robotic arms, kinematic data regarding movement of the plurality of robotic arms, and a topographical three-dimensional representation of anatomy in the surgical field captured by a second camera on another one of the plurality of robotic arms.

Illustrative Embodiment 14. The method of any of Illustrative Embodiments 11-13, further comprising providing the surgical simulation model with one or more of the following items: data from an electronic health record of the patient, data from an intraoperative sensor, imaging data of the patient, and previous video data of the patient.

Illustrative Embodiment 15. The method of any of Illustrative Embodiments 11-14, further comprising: in response to receiving user approval of the simulated performance of the surgical task, providing the signals generated in response to user-manipulation of the plurality of user input devices to a robotic arm controller to cause the plurality of robotic arms to perform the surgical task on the patient.

Illustrative Embodiment 16. A robotic surgical system comprising: a plurality of user input devices; a plurality of robotic arms; means for providing a surgical simulation model with input about a surgical field of a patient and signals representing user-manipulation of the plurality of user input devices to perform a surgical task on the patient; and means for displaying a video generated by the surgical simulation model that represents a simulated performance of the surgical task.

Illustrative Embodiment 17. The robotic surgical system of Illustrative Embodiment 16, wherein the surgical simulation model uses a generalized adversarial network and a variational autoencoder.

Illustrative Embodiment 18. The robotic surgical system of any of Illustrative Embodiments 16-17, wherein the input about the surgical field comprises one or more of the following items: a plurality of video frames of the surgical field captured by a first camera on one of the plurality of robotic arms, kinematic data regarding movement of the plurality of robotic arms, and a topographical three-dimensional representation of anatomy in the surgical field captured by a second camera on another one of the plurality of robotic arms.

Illustrative Embodiment 19. The robotic surgical system of any of Illustrative

Embodiments 16-18, further comprising means for providing the surgical simulation model with one or more of the following items: data from an electronic health record of the patient, data from an intraoperative sensor, imaging data of the patient, and previous video data of the patient.

Illustrative Embodiment 20. The robotic surgical system of any of Illustrative Embodiments 16-19, further comprising means for, in response to receiving user approval of the simulated performance of the surgical task, providing the signals generated in response to user-manipulation of the plurality of user input devices to a robotic arm controller to cause the plurality of robotic arms to perform the surgical task on the patient.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

What is claimed is:

1. A robotic surgical system comprising:
a user console comprising a display device and a plurality of user input devices;
a plurality of robotic arms;
a robotic arm controller configured to move the plurality of robotic arms in response to signals received from the user console that are generated in response to user-manipulation of the plurality of user input devices; and
a processor configured to:
provide a surgical simulation model with input about a surgical field of a patient;
provide signals generated in response to user-manipulation of the plurality of user input devices to perform a surgical task on the patient to the surgical simulation model instead of to the robotic arm controller; and
display, on the display device of the user console, a video generated by the surgical simulation model that represents a simulated performance of the surgical task.

2. The robotic surgical system of claim 1, wherein the surgical simulation model uses a generalized adversarial network and a variational autoencoder.

3. The robotic surgical system of claim 1, wherein the input about the surgical field comprises one or more of the following items: a plurality of video frames of the surgical field captured by a first camera on one of the plurality of robotic arms, kinematic data regarding movement of the plurality of robotic arms, and a topographical three-dimensional representation of anatomy in the surgical field captured by a second camera on another one of the plurality of robotic arms.

4. The robotic surgical system of claim 1, wherein the processor is further configured to provide the surgical simulation model with one or more of the following items: data from an electronic health record of the patient, data from an intraoperative sensor, imaging data of the patient, and previous video data of the patient.

5. The robotic surgical system of claim 1, wherein the processor is further configured to select the surgical simulation model from a plurality of surgical simulation models based information about a surgical procedure and/or the patient.

6. The robotic surgical system of claim 1, wherein the processor is further configured to provide an indication of a likelihood of success of the surgical task.

7. The robotic surgical system of claim 1, wherein the processor is further configured to:
in response to receiving user approval of the simulated performance of the surgical task, provide the signals generated in response to user-manipulation of the plurality of user input devices to the robotic arm controller to cause the plurality of robotic arms to perform the surgical task on the patient.

8. The robotic surgical system of claim 1, wherein the user console is on Earth, and the plurality of robotic arms are not on Earth.

9. The robotic surgical system of claim 1, wherein the user console is in one building on Earth, and the plurality of robotic arms are in another building on Earth.

10. The robotic surgical system of claim 1, wherein the processor is integrated in the user console.

11. A method comprising:
performing the following in a robotic surgical system comprising a plurality of robotic arms, the method comprising:
providing a surgical simulation model with input about a surgical field of a patient;
providing signals generated in response to user-manipulation of a plurality of user input devices to perform a surgical task on the patient to the surgical simulation model; and
displaying a video generated by the surgical simulation model that represents a simulated performance of the surgical task.

12. The method of claim 11, wherein the surgical simulation model uses a generalized adversarial network and a variational autoencoder.

13. The method of claim 11, wherein the input about the surgical field comprises one or more of the following items: a plurality of video frames of the surgical field captured by a first camera on one of the plurality of robotic arms, kinematic data regarding movement of the plurality of robotic arms, and a topographical three-dimensional representation of anatomy in the surgical field captured by a second camera on another one of the plurality of robotic arms.

14. The method of claim 11, further comprising providing the surgical simulation model with one or more of the following items: data from an electronic health record of the patient, data from an intraoperative sensor, imaging data of the patient, and previous video data of the patient.

15. The method of claim 11, further comprising:
in response to receiving user approval of the simulated performance of the surgical task, providing the signals generated in response to user-manipulation of the plurality of user input devices to a robotic arm controller to cause the plurality of robotic arms to perform the surgical task on the patient.

16. A robotic surgical system comprising:
a plurality of user input devices;
a plurality of robotic arms;
means for providing a surgical simulation model with input about a surgical field of a patient and signals representing user-manipulation of the plurality of user input devices to perform a surgical task on the patient; and
means for displaying a video generated by the surgical simulation model that represents a simulated performance of the surgical task.

17. The robotic surgical system of claim 16, wherein the surgical simulation model uses a generalized adversarial network and a variational autoencoder.

18. The robotic surgical system of claim 16, wherein the input about the surgical field comprises one or more of the following items: a plurality of video frames of the surgical field captured by a first camera on one of the plurality of robotic arms, kinematic data regarding movement of the plurality of robotic arms, and a topographical three-dimensional representation of anatomy in the surgical field captured by a second camera on another one of the plurality of robotic arms.

19. The robotic surgical system of claim 16, further comprising means for providing the surgical simulation model with one or more of the following items: data from an electronic health record of the patient, data from an intraoperative sensor, imaging data of the patient, and previous video data of the patient.

20. The robotic surgical system of claim 16, further comprising means for, in response to receiving user approval of the simulated performance of the surgical task, providing the signals generated in response to user-manipulation of the plurality of user input devices to a robotic arm controller to cause the plurality of robotic arms to perform the surgical task on the patient.
